(19) United States
(12) Patent Application Publication
(10) Pub. No.: US 2023/0405195 A1
(43) Pub. Date: Dec. 21, 2023

(54) DEVICE FOR PREPARING A DIALYSIS CONCENTRATE

(71) Applicant: VIVONIC GMBH, Sailauf (DE)

(72) Inventors: Patrick BESSLER, Erlenbach (DE); Stefan EBERLEIN, Höchberg (DE); Andreas HEMM, Alzenau (DE)

(73) Assignee: VIVONIC GMBH, Sailauf (DE)

(21) Appl. No.: 18/247,296

(22) PCT Filed: Sep. 30, 2021
(86) PCT No.: PCT/EP2021/076909

§ 371 Date: Mar. 30, 2023

(30) Foreign Application Priority Data
Oct. 1, 2020 (DE) 10 2020 125 648.5

(51) Int. Cl. A61M 1/16

(52) U.S. Cl. CPC A61M 1/1666

(57) ABSTRACT

The present invention relates to a device for preparing a dialysis concentrate, wherein the device has an inflow for the solvent and a receptacle for a raw concentrate substance to be dissolved by means of the solvent or miscible with the solvent and having a mixing container for mixing the solvent and the raw concentrate substance, wherein the device further has a controller that has a parameter unit that is configure to receive or determine at least one input parameter value, an optimization unit that is configured to determine an optimization of the mixing process based on the at least one input parameter value, and a control or regulation unit that is configured to control or regulate the preparation process of the device by means of one or more starting parameter values on the basis of the determined optimization.

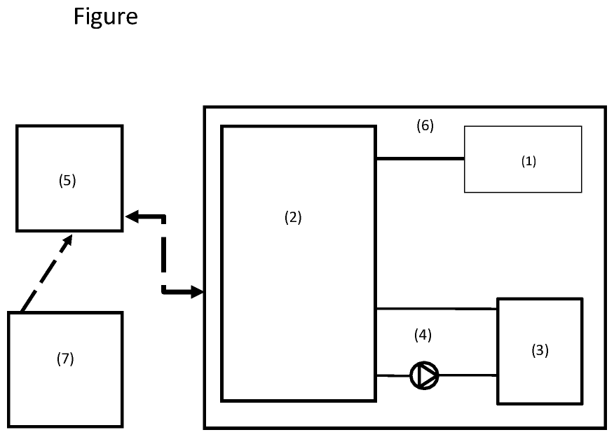

The present invention relates to a device for preparing a dialysis concentrate, wherein the device has an inflow for solvent and a receptacle for a raw concentrate substance to be dissolved by means of the solvent or to be mixed with the solvent and has a mixing container for mixing the solvent and raw concentrate substance.

It is known from the prior art to prepare dialysis concentrates from one or more raw concentrate substances and water or dialysis water in accordance with a largely strictly predefined mixing process. In this respect, this mixing process is typically adapted such that the worst basic conditions are assumed. They are, for example, cold dialysis water, a cold environmental temperature, a real time concentrate requirement, etc. This makes the use of large amounts of resources necessary such as thermal and mechanical energy and time.

The main parameter that have an influence on the mixing process are:
1. Mechanical energy such as is introduced by stirring, the operation of pumps, etc.;
2. Thermal energy such as the dialysis water temperature, the mixing temperature, the operation of a heating device, etc.; and
3. Time in the form of the mixing time.

The basic conditions of dialysis water temperature, environmental temperature, requirement time, etc. are in turn again dependent on a plurality of further basic conditions.

There are regular, predictable fluctuations therein. Temperatures frequently fluctuate over the course of the day due, for example, to the waste heat of other devices, dialysis water consumption, etc., over the course of a week (medium term heating effects), and over the seasons, e.g. due to environmental temperatures, the mains water temperature, etc.

There are also irregular, but sometimes also predictable fluctuations such as the weather that plays a role, for example, in the hot water production by means of solar energy for the admixture of input water and regulation of the dialysis water temperature or by means of heat exchangers.

A process known from the prior art for preparing a dialysis concentrate will be described in the following with reference to the FIGURE, with only the bordered region of the FIGURE being known from the prior art.

Dialysis water is metered in a mixing container 2 of the mixing system 6 by means of the dialysis water inflow 1. The raw concentration substance is furthermore metered in manually or automatically in individual components or premetered units. A mixing of raw concentrate substance with dialysis water and thus the preparation of the dialysis concentrate takes place in the mixing container 2 by one or more arbitrary mixing processes such as stirring, circulation or any other desired mixing processes. The dialysis concentrate under preparation is supplied to the measuring device 3 by means of the pump 4 for the purposes of quality control during the mixing process for a process control or for release prior to the termination of the mixing process. It is possible that a reference fluid such as dialysis water is supplied to the measuring device prior to and/or after each of these mixing processes so that an automatic process for the revalidation or calibration of the measuring device 3 can take place.

It is the underlying object of the present invention to improve a device and a method for the preparation of dialysis concentrate such that the preparation of the dialysis concentrate takes place in a resource-saving manner.

This object is achieved by a device having the features of claim 1 and by a method having the features of claim 9 and furthermore by a system having the features of claim 14.

Provision is made in accordance with the invention that the device comprises a controller or a controller unit that comprises the following:
a parameter unit that is configured for receiving or determining at least one input parameter value;
an optimization unit that is configured for the determination of an optimization of the mixing process from raw concentrate substance and solvent on the basis of the at least one input parameter value;
and a control or regulation unit that is configured to control or regulate the preparation process of the device by means of one or more starting parameter values based on the determined optimization.

In accordance with the invention, it is thus not always the same predefined preparation process, i.e. mixing process, that is carried out to prepare dialysis concentrate. Provision is rather made that one or more input parameters are considered such as one or more environmental parameters such as the temperature, etc. and that an optimization of the preparation process is carried on the basis thereof. The preparation process is controlled or regulated by means of one or more starting parameters such as the duration of the mixing process, its start, etc. If regulation is present, the controller is configured to perform the mixing process such that a desired value is reached wherever possible such as a specific desired duration, specific desired costs, or a specific capacity.

The parameter unit can be configured to obtain the at least one input parameter value from a sensor, from a database, or from another data source or by a manual input by the user. The parameter unit can thus receive either one or more input parameter values such as from one or more sensors such as temperature sensors or by input by a user. In the latter case, the parameter unit thus has an interface for a user such as a keyboard. It is also possible that the parameter unit is configured to determine the input parameter or parameters itself such as on the basis of historic values or generally by a prediction, e.g. by predicting the environmental temperature.

The device can include a measuring device such as a sensor for determining at least one input parameter value. It can here e.g. be the temperature of the environmental air, of the supplied dialyzate water, etc. or of the solution located in the mixing container. In the last case, the measuring device can preferably be connected to the mixing container by a line system provided with a pump.

Provision is made in a further embodiment of the invention that the inflow for the solvent is a dialyzate water inflow. Dialyzate water is preferably water that has been purified by a filtration process, with the water representing the permeate of the filter.

The receptacle for the raw concentrate substance can be formed by the mixing container itself. In this case, the raw concentrate substance to be dissolved or mixed is presented in the mixing container. It is also conceivable that the receptacle is formed by a component of the device different from the mixing container.

The raw concentrate substance can be a dry concentrate or a liquid concentrate or a slurry-like raw concentrate substance.

The “mixing of a raw concentrate substance with a solvent” is thus to be understood purely as a mixing process and/or dissolving process.

The optimization unit is preferably configured such that it carries out the optimization of the mixing process by a computer aided analysis process and/or optimization process. Neural networks, correlations, machine learning, etc. can be named as examples.

The controller can be configured to influence or provide one or more of the following starting parameters in the control or regulation: time of the dialysis water metering, time duration between the metering and the mixing process, time of the initiation of the mixing process, duration of the mixing process, proportions of mechanical, electrical, and/or thermal energy. These or other starting parameters are then applied to the mixing process.

The parameter unit can be configured to receive a desired value for an optimization parameter to be optimized, with the controller furthermore being configured to control or regulate the mixing process such that the desired value is obtained completely or as part of a specific deviation. Conceivable optimization parameters are the costs of the preparation, the time duration of the preparation, or the capacity. In the last case, an optimization is performed to the extent of how much dialysis water is available and/or when a requirement time for the dialysis concentrate has been reached.

In a further embodiment of the present invention, the parameter unit is configured to record or to determine a sensor value and/or a value input by the user and/or a value determined by prediction as the input parameter value.

The present invention further relates to a method of preparing a dialysis concentrate, wherein the method comprises the mixing of a raw concentrate substance with a solvent in which the raw concentrate substance is soluble or is miscible with the raw concentrate substance and wherein an optimization of the mixing process is performed on the basis of one or more input parameter values.

The optimization can here be performed as a control or as a regulation.

The input parameter can be a measured value, a value input by a user, or a determined value. The determined value can, for example, be a value determined or forecast by prediction.

The optimization preferably delivers one or more starting parameter values that are then used as part of the preparation of the dialysis concentrate.

The starting parameters can form one or more of the following parameters in an embodiment: time of the dialysis water metering, time duration between the metering and the mixing process, time of the initiation of the mixing process, duration of the mixing process, proportions of mechanical, electrical, and/or thermal energy.

The method in accordance with the invention is preferably carried out by means of a device in accordance with the Invention.

The present invention further relates to a system comprising a device in accordance with one of the claims 1 to 8 and to a blood treatment device, wherein the device is a component of the blood treatment device or is in fluidic communication therewith.

The blood treatment device is preferably a dialysis machine.

It is pointed out at this point that the terms “a” and “one” do not necessarily refer to exactly one of the elements, even though this represents a possible embodiment, but can also designate a plurality of the elements. The use of the plural equally also includes the presence of the element in question in the singular and, conversely, the singular also includes a plurality of the elements in question.

Further advantages and particulars of the invention will be explained in more detail with reference to an embodiment shown in the FIGURE.

The FIGURE shows a device for preparing dialysis concentrate in accordance with the invention.

Differing from the device known from the prior art, the mixing system 6 already described in more detail above by way of example is connected to a controller 5 or the controller 5 is a component of the mixing system 6. The controller 5 is configured to carry out a detection and analysis of measured values and to influence the mixing process or to optimize it based thereon.

The mixing process optimized by the controller 5 is then implemented by the mixing system 6 by means of one or more starting parameter values.

The controller 5 stands generally for any desired hardware based and/or software based hardware and/or software unit.

The controller 5 can be configured to receive data of the mixing system 6 and/or other data such as data of the surroundings or environmental data that are marked by reference numeral 7 in the FIGURE. In this respect, it can be the dialysis water temperature, the environmental temperature, the temperature of the solution, and/or data of other systems, etc.

The controller 6 collects and processes these data. It determines the optimum mixing process for preparing a dialysis concentrate based on these data.

The controller 6 optimizes the mixing process by means of one, more, or all the control options. These control options, i.e. starting parameter values, can, for example, be one or more of the following points:
time of the dialysis water metering (phases of elevated dialysis water temperature, energy costs, capacity);
pause between the metering and initiation of the mixing process (use of the environmental temperature);
time of the mixing process (energy costs); and
optimizing the portions of mechanical energy, electrical energy, thermal energy, and/or mixing duration in the mixing process.

The controller 6 can be configured to optimize the mixing process with respect to one or more parameters. One or more of the following target values are thus conceivable as desired values:
costs;
time; and
capacity (availability of the dialysis water and/or requirement time for the dialysis concentrate).

The controller can be configured to forecast the basic conditions for the mixing process and to control the mixing process on the basis of the forecast. This forecast can take place, for example, on the basis of the following data:
historical data such as when how much dialysis water is consumed;
predicted data such as the weather forecast having an influence on the inflow water temperature and/or having an influence on the environmental temperature;
daily profiles of the dialysis water temperature and/or of the environmental temperature, e.g. by water consumption values, heating control, individual profiles of the treatment centers;
weekly profiles of the dialysis water temperature and/or of the environmental temperature (by medium term changes of the heating control, individual profiles of the treatment centers);
seasonal profiles; and
information on different raw concentrate substances such as the grain size, different compositions of the individual components of the raw concentrate substance.

Input parameters or their values for the optimization process such as further environmental data can be transferred to the controller via an interface. It is possible here that the controller is configured such that the input parameter values are input via a user interface or are determined and/or provided by a sensor. It is alternatively or additionally possible that the data are used from any desired databases or data sources via an interface. Input parameter values from historical and/or current values and/or from prediction systems for the future that provide specific future values by prediction are, for example, conceivable.

Alternatively or additionally, the values can be historical, current, or estimated values or sensor signal such as temperature, pressure, consumption, prices, and availability of energy, media properties, composition, particle size, properties of the starting substance or substances, etc.

One or more optimization targets such as the required time of the completion of the concentrate, the energy consumption for the concentrate preparation, the time or duration of the preparation process, the total costs for the preparation, etc. can be transferred to the controller via an interface.

Based on this, the controller determines the parameters and their values of the preparation process by means of which the objective can be reached and controls the mixing process accordingly.

A regulation is also conceivable, and indeed such that one or more values are fed back to the controller during the preparation process and the controller carries out a comparison between the actual value and the desired value. If a difference is determined, the controller can be configured to influence the preparation process, i.e. the mixing process, such that the difference is minimized or adopts the value zero.

The controller 5 can, for example, carry out an optimization of the mixing process of the mixing and preparation system by means of computer assisted analysis processes and/or optimization processes such as correlations, neural networks, machine learning, etc. It can here, for example, be the extension or cutting of the mixing time, the adaptation of the temperature during the mixing process, or the control and supply of energy such as mechanical energy to the mixing process. The controller 5 can thus evaluate and ensure the required quality of the dialysis concentrate.

An exemplary routine of a preparation process in accordance with the invention will be explained in the following:

There is first the attachment of all the raw materials required for the preparation of the dialysis concentrate such as fluids, solvents, raw substances, mixes, etc.

The controller 5 receives one or more data or input parameters, i.e. actual values of parameter values, either by input or by invocation. They can be data from databases, manually input data, or sensor data. Exemplary data are:
historical data such as when how much dialysis water is consumed and/or what proportions circulate in a ring line system and thus have a different energy content than freshly produced dialysis water;
predicted data such as weather forecasts having an influence on the or temperature of the inflow water or having an influence on the environmental temperature;
daily profiles of the dialysis water temperature and/or of the environmental temperature, e.g. by water consumption values, heating control, individual profiles of the treatment centers;
weekly profiles of the dialysis water temperature and/or of the environmental temperature based on medium term changes of the heating control, individual profiles of the treatment centers;
seasonal profiles;
information on different raw concentration substances such as the grain size of the powder or granulate that is to be dissolved and/or their composition from individual substances; and
prices and/or availability of energy.

The controller can carry out the optimization process by means of computer assisted analysis processes such as correlations, neural networks, machine learning, etc. with respect to one or more predetermined targets such as costs, time or duration, capacity (e.g. availability of dialysis water and/or of the requirement time of the dialysis concentrate).

One or more of the parameters named in the following can be used for the optimization, for example:
time of the dialysis water metering (phases of elevated dialysis water temperature, energy costs, capacity);
pause between the metering and initiation of the mixing process (use of the environmental temperature);
time of the mixing process (energy costs); and
optimizing the portions of mechanical energy, electrical energy, thermal energy, and/or mixing duration in the mixing process.

A transfer of the parameter or parameters optimized for the mixing process or of the starting parameter values then takes place to the mixing system 6 or the controller 5 performs the mixing process on the basis of these parameters. The mixing process preferably takes place by control or regulation by means of the controller 5. The mixing process takes place while using the optimized parameters, with the mixing process preferably taking place as described initially on the prior art.

Advantages of the present invention comprise the parameters of the preparation process for the dialysis concentrate no longer having to be adapted to the worst case such as low temperatures, high energy costs, etc. In accordance with the invention, an adaptation of the parameter or parameters of the preparation process for the dialysis concentrate rather takes place adapted to the historical, current, or future circumstances so that resources can be saved.

Furthermore in accordance with the invention not only the mixing process can be adapted to the historical, current, or future circumstances. It is also possible to mix the mixing process with a process specifically adapted to this situation in phases of optimum basic conditions such as temperature, etc.

A further optimization of the total process to enable the dialysis treatment and a saving of resources associated therewith is possible by the communication between different devices such as the heating, a dialysis machine, or the dialysis water consumption, or the reverse osmosis system and by an adaptive regulation of the metering and mixing process by the controller 5. The same applies accordingly to the optimization of the mixing process by computer assisted analysis and regulation processes.

1. A device for preparing a dialysis concentrate, wherein the device has an inflow for the solvent and a receptacle for a raw concentrate substance to be dissolved by means of the solvent or miscible with the solvent and has a mixing container for mixing the solvent and the raw concentrate substance, characterized in that the device further has a controller that has a parameter unit that is configured to receive or determine at least one input parameter value, an optimization unit that is configured to determine an optimization of the mixing process based on the at least one input parameter value, and a control or regulation unit that is configured to control or regulate the preparation process of the device by means of one or more starting parameter values on the basis of the determined optimization.

2. A device in accordance with claim 1, characterized in that the parameter unit is configured to obtain the at least one input parameter value from a sensor, from a data source, or by manual input directly or by calculation.

3. A device in accordance with claim 1, characterized in that the device contains a measuring device for determining at least one input parameter value of the solution located in the mixing container, with the measuring device preferably being connected to the mixing container by a line system provided with a pump.

4. A device in accordance with claim 1, characterized in that the inflow for the solvent is a dialysis water inflow; and/or in that the receptacle for the raw concentrate substance is formed by the mixing container itself or by a component of the device different from the mixing container.

5. A device in accordance with claim 1, characterized in that the optimization unit is configured to carry out the optimization of the mixing process by a computer assisted analysis process and/or optimization process.

6. A device in accordance with claim 1, characterized in that the controller is configured to influence or provide one or more of the following starting parameters in the control or regulation. time of the dialysis water metering, time duration between the metering and the mixing process, time of the initiation of the mixing process, duration of the mixing process, proportions of mechanical, electrical, and/or thermal energy.

7. A device in accordance with claim 1, characterized in that the parameter unit is configured to receive a desired value for an optimization parameter to be optimized; and in that the controller is furthermore configured to control or regulate the mixing process such that the desired value is obtained completely or as part of a specific deviation.

8. A device in accordance with claim 1, characterized in that the parameter unit is configured to record or to determine a sensor value and/or a value input by the user and/or a value determined by prediction and/or historical data as the input parameter value.

9. A method of preparing a dialysis concentrate, wherein the method comprises the mixing of a raw concentrate substance with a solvent in which the raw concentrate substance is soluble or is miscible with the raw concentrate substance, characterized in that an optimization of the mixing process is performed on the basis of one or more input parameter values.

10. A method in accordance with claim 9, characterized in that the optimization is carried out as a control or as a regulation; and/or in that the input parameter value is a measured value, a value input by the user, or a determined value.

11. A method in accordance with claim 9, characterized in that the result of the optimization forms one or more starting parameter values.

12. A method in accordance with claim 11, characterized in that the starting parameters form one or more of the following parameters: time of the dialysis water metering, time duration between the metering and the mixing process, time of the initiation of the mixing process, duration of the mixing process, proportions of mechanical, electrical, and/or thermal energy.

13. A method in accordance with claim 10, characterized in that the determined value is a forecast value.

14. A system comprising a device in accordance with claim 1 and a blood treatment device, wherein the device is a component of the blood treatment device or is in fluidic communication therewith.

15. A system in accordance with claim 14, characterized in that the blood treatment device is a dialysis device.
